(19) United States
(12) Patent Application Publication
(10) Pub. No.: US 2026/0241112 A1
(43) Pub. Date: Aug. 20, 2026

(54) COMFORTABLE SYRINGE

(71) Applicant: Tae Heung IM, Chungcheongbuk-do (KR)

(72) Inventor: Mun Su LEE, Chungcheongbuk-do (KR)

(21) Appl. No.: 19/159,839

(22) PCT Filed: Mar. 21, 2024
(86) PCT No.: PCT/KR2024/003580

§ 371 Date: Aug. 26, 2025

(30) Foreign Application Priority Data
Mar. 21, 2023 (KR) 10-2023-0036254

(51) Int. Cl. A61M 5/32

(52) U.S. Cl. CPC A61M 5/32; A61M 2205/0216; A61M 2205/59; A61M 2207/00

(57) ABSTRACT

The present invention relates to a syringe, and a comfortable syringe according to the present invention comprises a cylinder having a needle coupled to a tip end and a cylinder space therein, a piston fitted into the cylinder space, and a load bar coupled to the piston, and the piston is moved by pulling the load bar to change the pressure within the cylinder, wherein the syringe comprises a sponge part having a rod shape extending a certain length in the longitudinal direction of the needle, being compressible and elastically restored when a compressive force is removed, and configured to wrap the needle so that the needle cannot be seen in the elastically restored state, and a needle passage hole formed through the center of the sponge part so that the needle can pass through.

### TECHNICAL FIELD

The present invention relates to a syringe, and more particularly, to a syringe that eliminates the fear of injections by making the needle invisible to the patient, thereby allowing the patient to receive an injection comfortably.

### BACKGROUND ART

When we are sick, we go to the hospital to get treatment. Depending on the doctor's diagnosis, oral medication or injections are prescribed. Injections are generally more effective than oral medication.

People are required to receive timely vaccinations against pathogens designated as Class 1 communicable diseases at birth, and annual influenza vaccines are also administered, so it is common for people to receive shots from a very young age.

However, even adults are afraid of needles, as they know in advance that a sharp needle piercing their body will be painful. Adults are like that, but infants and children are bound to have even more resistance. Infants and children are also very scared because they know what a long, sharp needle is, and they often burst into tears just by going to the hospital entrance. Doctors try to calm people down by saying it will be over soon and that it won't hurt, but once a person sees the sharp needle, it's hard to erase the fear from their mind.

The skin has three layers: the epidermis, the dermis, and the subcutaneous fat. The outermost layer is the epidermis, the middle is the dermis, and the inner layer is the subcutaneous fat. The layer where pain is felt is the epidermis. Therefore, if a needle passes quickly through the epidermis, pain is not felt. So, in Oriental medicine, when inserting a needle, they put it in a needle container and flick it with a finger to quickly pass it through the epidermis so that you don't feel the pain, and since the needle is hidden in the needle container before inserting it, there is less rejection.

However, there was a problem with injections using syringes in existing hospitals, as the sharp needle was visible to the patient, causing the patient to refuse the injection or show an excessive reaction.

### DETAILED DESCRIPTION OF THE INVENTION

### Technical Problem

The present invention has been devised to solve the problems of the prior art, and the purpose of the present invention is to provide a comfortable syringe that makes the sharp needle invisible to the patient's eyes, thereby freeing the patient from fear caused by visual information, and significantly alleviates the aversion to injections by using the soft feel and comfortable appearance of a sponge instead of a sharp needle.

### Technical Solution

In order to achieve the above-described purpose, a comfortable syringe according to the present invention comprises a cylinder having a needle coupled to a tip end and a cylinder space therein, a piston fitted into the cylinder space, and a load bar coupled to the piston, wherein the syringe changes the pressure within the cylinder by pulling the load bar to move the piston, the syringe comprises a sponge portion having a rod shape extending a certain length in the longitudinal direction of the needle, being compressible and elastically restored when a compressive force is removed, and configured to wrap the needle so that the needle cannot be seen in the elastically restored state, and a needle passage hole formed through the center of the sponge portion so that the needle can pass through.

In addition, it is preferable that a double-sided adhesive portion is provided between the sponge portion and the tip portion of the cylinder so that the sponge portion is connected to the tip portion of the cylinder.

In addition, it is preferable to further include a thread for preventing detachment, which is connected at both ends to the double-sided adhesive portion and passes through the inside of the sponge portion so that the sponge portion is threaded.

### Advantageous Effects

As described above, the comfortable syringe according to the present invention has the advantage of preventing the patient from fearing pain in advance by seeing a sharp needle because the needle is not visible to the patient, and also reducing the aversion to injections in young patients by providing a comfortable appearance and a soft feeling when touched by the sponge, thereby facilitating a smooth treatment process and increasing the treatment effect, thereby reducing the burden on medical personnel.

### DESCRIPTION OF DRAWINGS

FIG. 1 is an exploded perspective view of a comfortable syringe according to the present invention.

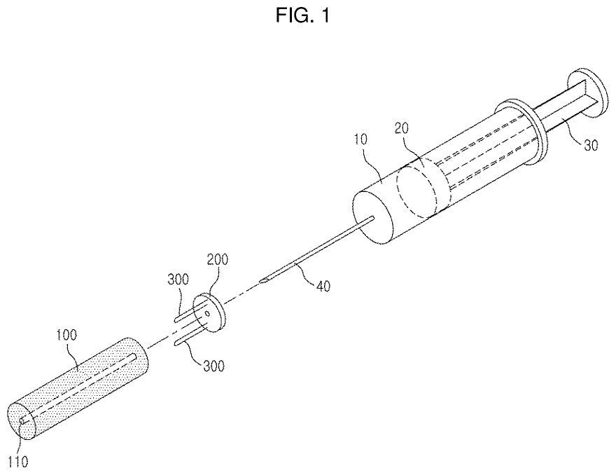

FIG. 2 is an assembled perspective view of a comfortable syringe according to the present invention.

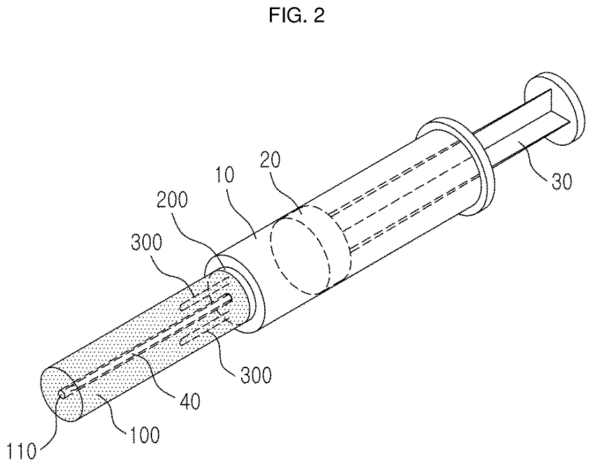

FIG. 3 is a diagram showing a comfortable syringe according to the present invention in use.

### DESCRIPTION OF EMBODIMENTS

Hereinafter, specific embodiments of the present invention will be described in detail with reference to the attached drawings.

The comfortable syringe according to the present invention comprises a cylinder (10), a piston (20), a load bar (30), an injection needle (40), a sponge portion (100), a needle passage hole (110), a double-sided adhesive portion (200), and a detachment prevention thread (300). The syringe comprises a cylinder (10) with a needle (40) attached to the tip and a cylinder space formed therein, a piston (20) fitted into the cylinder space, and a load bar (30) connected to the piston (20). The configuration of the syringe, which changes the pressure within the cylinder (10) by pulling the load bar (40) and moving the piston (20), is identical to that of conventional syringes.

The sponge portion (100) surrounds the injection needle (40) to make it invisible from the outside, has a length that can completely cover the injection needle (40), and has a circular rod shape that extends along the length of the needle (40). It is made of a sponge material, so it is easy to be deformed by an external force, and in addition, it has a property of elastically recovering its original shape when the external force is removed, so it is compressible, and when the compressive force is removed, it elastically recovers, and in the elastically restored state, it wraps around the injection needle (40) to make the needle (40) invisible. In other words, the injection needle (40) is hidden inside the sponge portion (100).

The needle passage hole (110) penetrating the center of the sponge portion (100) is a passage that passes through the center of both flat end surfaces of the sponge portion (100) in a straight line, and when the sponge portion (100) is not compressed, the injection needle (40) is positioned inside the needle passage hole (110).

The double-sided adhesive portion (200) is a double-sided adhesive tape in the shape of a circular plate with a hole through which a needle (40) passes in the center. One side of the double-sided adhesive portion (200) is adhered to the outer surface of the tip of the cylinder (10) of the syringe, and the opposite side of the double-sided adhesive portion (200) is adhered to one end surface of the sponge portion (100), so that one end surface of the sponge portion (100) is coupled to the syringe.

The detachment prevention thread (300) is provided because the double-sided adhesive portion (200) alone may not completely eliminate the risk of the sponge portion (100) coming off from the syringe. The knots at both ends of the detachment prevention thread (300) are fixed between the side of the double-sided adhesive portion (200) on which the cylinder (10) of the syringe is located and the double-sided adhesive portion (200), and the remaining part of the detachment prevention thread (300) passes through the double-sided adhesive portion (200) and then passes through the body of the sponge portion (100), so that the sponge portion (100) is threaded to the detachment prevention thread (300), thereby preventing detachment of the sponge portion (100).

It is preferable that the detachment prevention thread (300) is provided on both sides with the injection needle (40) at the center.

The operation of the comfortable syringe according to the present invention configured as described above is described as follows.

When the syringe needle (40) is stored with the sponge portion (100) covered and the syringe needle (40) is inserted into the vial to fill the medicine, the sponge portion (100) covering the syringe needle (40) shrinks significantly reducing its volume.

After taking out an appropriate amount of medicine from the vial and removing the needle (40) from the vial, the sponge portion (100) relaxes and returns to its original shape, wrapping around the needle (40) again so that the needle (40) becomes invisible.

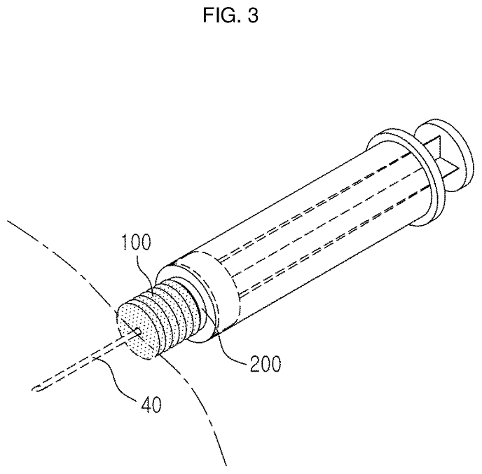

Then, the syringe containing the medicine is brought to the patient, and the syringe is pushed while the end of the sponge portion (100) is placed against the patient's body, so that the injection needle (40) is inserted into the patient's body, and in this state, the load bar (30) is pushed to inject the medicine. At this time, as shown in FIG. 3, the sponge portion (100) shrinks and contracts, so that the volume is significantly reduced.

During this process, the injection needle (40) becomes invisible to the patient's eyes, and only the sponge portion, which gives off a soft feeling, is visible, so that even young patients can comfortably undergo the injection process. Considering that conventional pediatric and adolescent care required the combined efforts of doctors, nurses, and parents to administer injections to young patients, the present invention can be of significant assistance in medical practice.

As described above, the present invention has been described only with respect to preferred specific examples. However, it is obvious to those skilled in the art that various modifications and variations are possible within the technical scope of the present invention based on the above specific examples, and it is also natural that such modifications and variations fall within the scope of the appended claims.

What is claimed is:

1. A comfortable syringe comprising a cylinder having a needle attached to the tip and a cylinder space within it, a piston fitted into the cylinder space, and a load bar coupled to the piston, wherein the piston is moved by pulling the load bar to change the pressure within the cylinder, the syringe comprising:
a sponge portion having a rod shape extending a predetermined length in the longitudinal direction of the needle, made of a sponge material, compressible, elastically recovering when compression is removed, and configured to wrap the needle so that the needle cannot be seen in the elastically restored state; and
a needle passage hole formed through the center of the sponge portion to allow the needle to pass through.

2. The comfortable syringe of claim 1, wherein a double-sided adhesive portion is provided between the sponge portion and the tip of the cylinder so that the sponge portion is connected to the tip portion of the cylinder.

3. The comfortable syringe of claim 2, wherein it further comprises a detachment prevention thread, the two ends of which are connected to the double-sided adhesive portion and which passes through the interior of the sponge portion so that the sponge portion is threaded.
